(19) United States
(12) Patent Application Publication
(10) Pub. No.: US 2012/0023647 A1
(43) Pub. Date: Feb. 2, 2012

(54) GOGGLES WITH LENS PROTECTION FILM

(76) Inventor: Soo An PARK, Seongnam-si (KR)

(21) Appl. No.: 13/051,783

(22) Filed: Mar. 18, 2011

(30) Foreign Application Priority Data
Jul. 27, 2010 (KR) 10-2010-0072534

(51) Int. Cl. A61F 9/02

(52) U.S. Cl. 2/438

(57) ABSTRACT

The present invention relates to goggles with lens protection film include: film adapted to cover the front surfaces of lenses; a film supply part adapted to be coupled to one side of the goggles so as to unroll the film toward the front of the lenses in a state of winding the film thereinto; a film return part adapted to be coupled to the other side of the goggles so as to roll the film unrolled to the front of the lenses thereinto; a drive part adapted to be coupled to the film return part so as to generate power to automatically roll the film; and a manipulating part adapted to be connected to the drive part with wire or without wire so as to remotely control an operation of the drive part.

### BACKGROUND OF THE INVENTION

1. Field of the Invention

The present invention relates to goggles with lens protection film that is capable of preventing foreign materials from sticking to the lenses thereof, thereby keeping a wearer's view from being obstructed.

2. Background of the Related Art

Generally, when people ski or ride bikes, they should wear goggles so as to protect their eyes. For example, when someone rides a trail bike on a rough road, he should wear goggles so as to prevent foreign materials like dust, lumps of clay and so on from invading his eyes.

The invasion of the foreign materials is prevented when the goggles are worn, but there occurs another problem that the foreign materials stick to the lenses of the goggles to cause a wearer's view to be obstructed.

In this case, the foreign materials sticking to the lenses may be removed simply with the wearer's hand or sleeve, but at this time, they spread widely on the lenses to cause his view to be more obstructed. During his riding, especially, if his hand holding a steering wheel of the bike is used temporarily to remove the foreign materials out of the lenses of the goggles, there occurs another problem that the bike falls down.

### SUMMARY OF THE INVENTION

Accordingly, the present invention has been made in view of the above-mentioned problems occurring in the prior art, and it is an object of the present invention to provide goggles with lens protection film that is capable of preventing foreign materials like dust, clay and so on sticking to lenses of the goggles while a wearer is skiing or is riding a bike and thus keeping the wearer's view from being obstructed.

It is another object of the present invention to provide goggles with lens protection film that is capable of preventing foreign materials sticking to lenses of the goggles from spreading widely when the foreign materials are cleaned by a wearer's hand during his skiing or riding and further preventing the wearer from falling down by the loss of balance during his skiing or riding because his hand is used to remove the foreign materials out of the lenses of the goggles.

To accomplish the above objects, according to the present invention, there are provided goggles having lenses protecting a wearer's eyes, a frame coupled to the lenses in such a manner as to be brought into close contact with the wearer's face, and a band coupled to both sides of the frame in such a manner as to be worn around the wearer's head, the goggles including: film made of a transparent material so as to provide a clear view in a state of covering the front surfaces of the lenses and having an elongated belt-like shape so as to be rolled; film supply part adapted to be coupled to one side of the goggles so as to unroll the film toward the front of the lenses in a state of winding the film thereinto; a film return part adapted to be coupled to the other side of the goggles so as to roll the film unrolled to the front of the lenses thereinto; a drive part adapted to be coupled to the film return part so as to generate power to automatically roll the film; and a manipulating part adapted to be connected to the drive part with wire or without wire so as to remotely control an operation of the drive part.

According to the present invention, preferably, each of the film supply part and the film return part includes: a case adapted to be coupled to one side of the goggles and having entrance and exit formed in an opposite direction thereto; a winding roll disposed inside the case and adapted to be rotated forwardly to roll or unroll the film thereonto or therefrom; and a one-way clutch adapted to prevent the winding roll from being rotated reversely.

According to the present invention, preferably, the one-way clutch has a latch gear disposed at the inside of the case and an elastic latch adapted to be coupled to the winding roll in such a manner as to be locked to the latch gear when the winding roll is rotated forwardly and unlocked from the latch gear when the winding roll is rotated reversely.

According to the present invention, preferably, the drive part has a motor adapted to be coupled to the winding roll and a receiver module adapted to receive a wireless signal and to operate the motor, and the manipulating part has a switch adapted to control the activation of the drive part and a transmitter module adapted to transmit the wireless signal in accordance with the manipulation of the switch.

According to the present invention, preferably, the goggles further include an auxiliary part adapted to be coupled to the film return part so as to roll the film in a manually operated manner.

According to the present invention, preferably, the auxiliary part includes: a reel shaft-coupled coaxially to the winding roll of the film return part; a spring disposed inside the reel and adapted to rotate the reel forwardly and reversely by means of the elastic force thereof; a pulling string adapted to be wound along the outer periphery of the reel and having a hand grip disposed at the end thereof; and a one-way clutch disposed between the reel and the winding roll of the film return part and adapted to rotate the winding roll in one direction.

According to the present invention, preferably, the one-way clutch includes: a latch gear disposed along the inner surface of the upper periphery of the winding roll; and an elastic latch adapted to be coupled to the reel in such a manner as to be locked to the latch gear when the reel is rotated forwardly and unlocked from the latch gear when the reel is rotated reversely.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view showing goggles with lens protection film according to a first embodiment of the present invention;

FIG. 2 is a plan sectional view showing the goggles with lens protection film according to the first embodiment of the present invention;

FIG. 3 is a sectional view taken along the line A-A of FIG. 2;

FIG. 4 is a sectional view taken along the line B-B of FIG. 2;

FIG. 5 is a sectional view taken along the line C-C of FIG. 4;

FIG. 6 is an exploded perspective view showing the main parts of the goggles with lens protection film according to the first embodiment of the present invention;

FIG. 7 is a side sectional view showing a manipulating part of the goggles with lens protection film according to the first embodiment of the present invention;

FIG. 8 is a perspective view showing goggles with lens protection film according to a second embodiment of the present invention;

FIG. 9 is a front sectional view showing the main parts of the goggles with lens protection film according to the second embodiment of the present invention;

FIG. 10 is a sectional view taken along the line D-D of FIG. 9; and

FIG. 11 is a sectional view taken along the line E-E of FIG. 9.

### DETAILED DESCRIPTION OF THE PREFERRED EMBODIMENT

Hereinafter, an explanation on goggles with lens protection film according to preferred embodiments of the present invention will be in detail given with reference to the attached drawings.

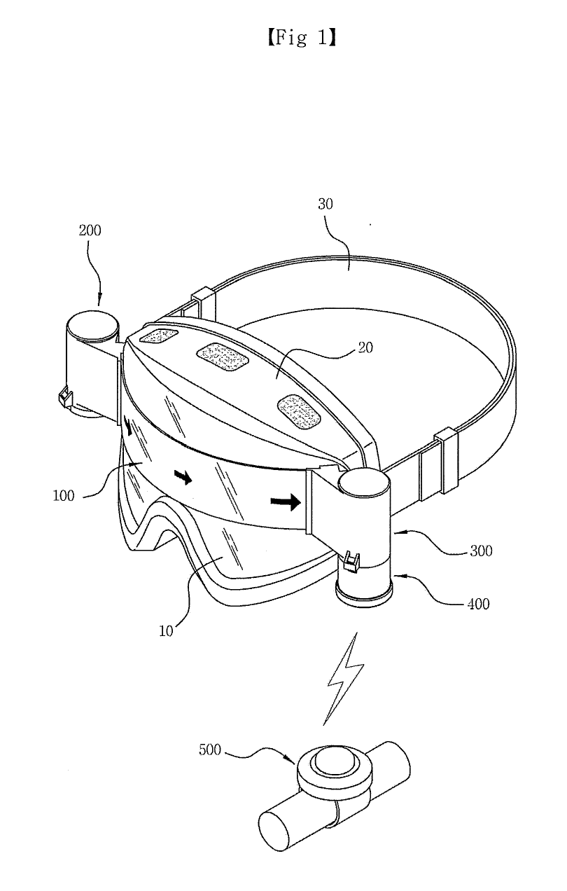

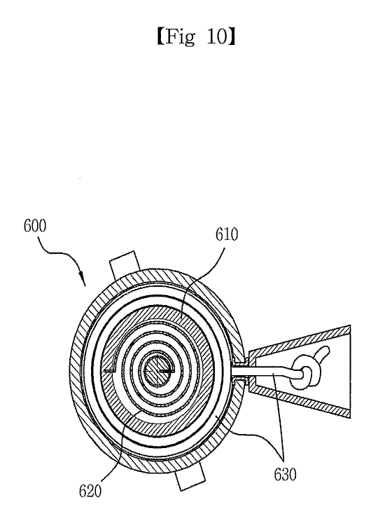

According to the present invention, as shown in FIGS. 1 to 10, goggles with lens protection film include film 100 adapted to cover the front surfaces of lenses 10, a film supply part 200 adapted to unroll the film 100 toward the front of the lenses 10 in a state of winding the film 100 thereinto; a film return part 300 adapted to roll the film 100 unrolled to the front of the lenses thereinto; a drive part 400 adapted to generate power to automatically roll the film 100; and a manipulating part 500 adapted to remotely control an operation of the drive part 400.

The film 100 is made of a transparent material so as to provide a clear view in the state of covering the front surfaces of the lenses 10 and has an elongated belt-like shape so as to be rolled. For example, the film 100 is made of a transparent synthetic resin and has a width in a range between 5 cm and 10 cm so as to provide a clean view.

The film supply part 200 is coupled to one side of the goggles and serves to unroll the film 100 toward the front of the lenses 10 in the state of winding the film 100 thereinto. The film supply part 200 may be coupled for example to one lens 10 or a frame 20 for goggles.

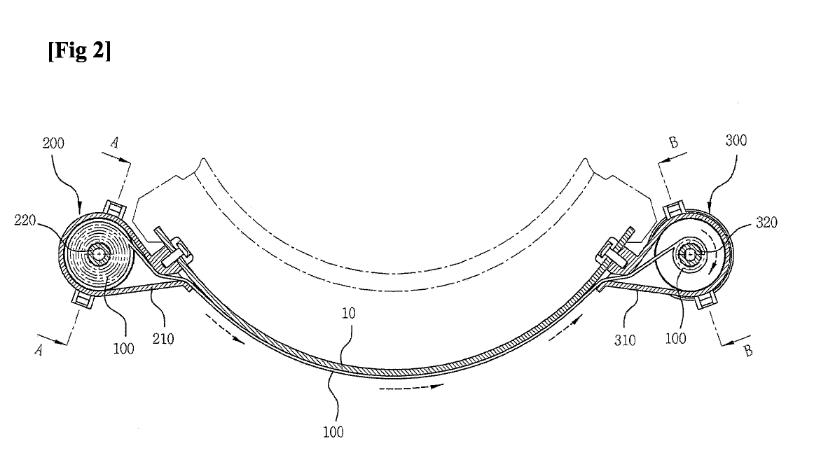

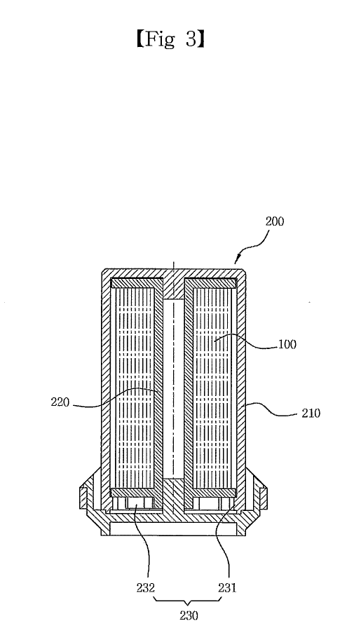

As shown in FIGS. 2 and 3, the film supply part 200 includes a case 210, a winding roll 220, and a one-way clutch 230. The case 210 is coupled to one side of the goggles and has entrance and exit formed in an opposite direction thereto. The entrance and exit of the case 210 is formed near one side lens 10 such that the film 100 is unrolled at a state of being brought into close contact with one side lens 10. The winding roll 220 is disposed inside the case 210 and is rotated forwardly to roll or unroll the film 100 thereonto or therefrom. The one-way clutch 230 serves to allow the winding roll 210 to be gently rotated forwardly to unroll the film 100 therefrom, while preventing the winding roll 210 from being rotated reversely to roll the film 100 thereonto.

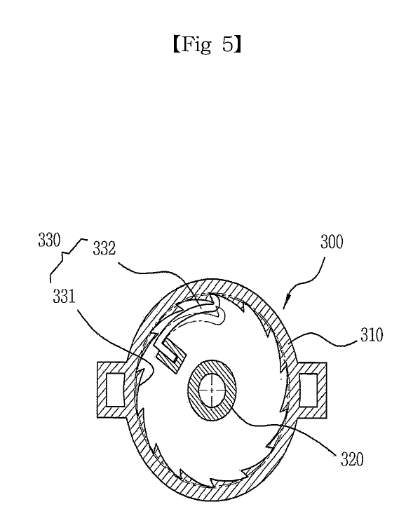

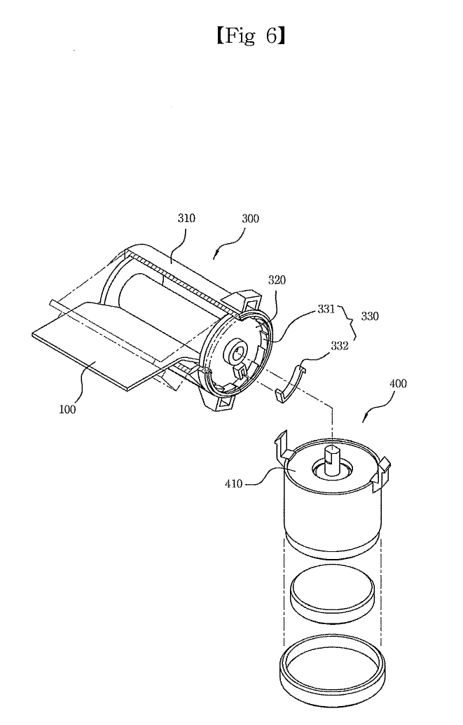

The one-way clutch 230 is composed of a latch gear 231 disposed at the inside of the case 210 and an elastic latch 232 adapted to be coupled to the winding roll 220. The elastic latch 232 is locked to the latch gear 231 when the winding roll 220 is rotated forwardly, and contrarily, it is unlocked from the latch gear 231 when the winding roll 220 is rotated reversely. For example, the configuration is the same as that in FIGS. 5 and 6.

The film return part 300 is coupled to the other side of the goggles and serves to roll the film 100 unrolled toward the front of the lenses 10 thereinto. The film return part 300 may be coupled for example to one lens 10 or the frame 20 for goggles.

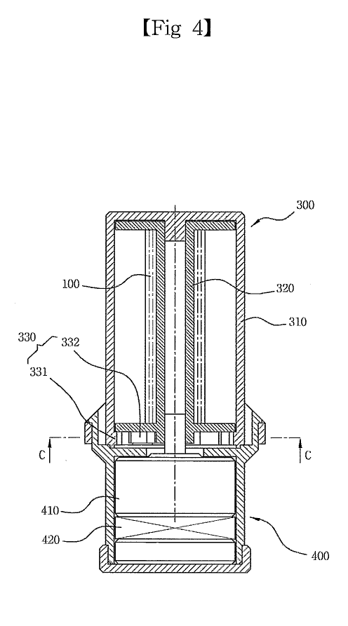

As shown in FIGS. 2 and 4, the film return part 300 includes a case 310, a winding roll 320, and a one-way clutch 330. The case 310 is coupled to the other side of the goggles and has entrance and exit formed at an opposite direction thereto. The entrance and exit of the case 310 is formed near one side lens 10 such that the film 100 is rolled in a state of being brought into close contact with one side lens 10. The winding roll 320 is disposed inside the case 310 and is rotated forwardly to roll or unroll the film 100 thereonto or therefrom. The one-way clutch 330 serves to allow the winding roll 310 to be gently rotated forwardly to roll the film 100 thereonto, while preventing the winding roll 310 from being rotated reversely to unroll the film 100 therefrom.

As shown in FIGS. 5 and 6, the one-way clutch 330 is composed of a latch gear 331 disposed at the inside of the case 310 and an elastic latch 332 adapted to be coupled to the winding roll 320. The elastic latch 332 is locked to the latch gear 331 when the winding roll 220 is rotated forwardly, and contrarily, it is unlocked from the latch gear 331 when the winding roll 320 is rotated reversely.

As mentioned above, the film supply part 200 serves to unroll the film 100 in the state of initially winding the film 100 thereinto, and the film return part 300 serves to roll the film 100 being unrolled in the state where the film 100 is not initially rolled thereinto. Accordingly, the film supply part 200 and the film return part 300 have a generally symmetrical configuration to each other.

The drive part 400 is adapted to be coupled to the film return part 300 and serves to generate power to automatically wind the film 100. The drive part 400 has a motor 410 adapted to rotate the film return part 300 so as to automatically wind the film 100, and therefore, the film 100 is wound not manually, but by means of the rotation of the film return part 300 through the motor 410.

The manipulating part 500 is connected to the drive part 400 with wire or without wire and serves to remotely control the operation of the drive part 400. The manipulating part 500 is connected to the drive part 400 with wire or without wire, and on the other hand, it is mounted on a hand grip of a stick if a wearer skies and on a steering wheel if he rides a bike, which enables the manipulation with his fingers, without separating his hand from the stick or the steering wheel.

The configuration where the drive part 400 is connected to the manipulating part 500 without wire will be explained below.

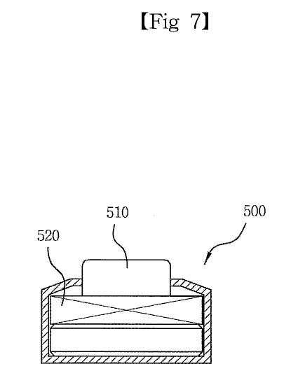

As shown in FIGS. 4 and 6, the drive part 400 has the motor 410 coupled to the winding roll 320 and a receiver module 420 adapted to receive a wireless signal and to operate the motor 410, and as shown in FIGS. 1 and 7, the manipulating part 500 has a switch 510 adapted to control the activation of the drive part 400 and a transmitter module 520 adapted to transmit the wireless signal in accordance with the manipulation of the switch 510. The drive part 400 includes a battery for actuating the motor 410 and for operating the receiver module 420, and the manipulating part 400 also includes a battery for operating the transmitter module 520.

Accordingly, if lots of foreign materials stick to the film 100 to cause the wearer's view to be obstructed, the switch 510 of the manipulating part 500 is pressed to transmit a signal through the transmitter module 520, and thus, the receiver module 410 of the drive part 400 receives the signal to rotate the motor 410. At this time, if the switch 510 is kept at the pressed state, the signal is kept by means of the transmitter module 520 and the receiver module 420 such that the motor 410 is continuously rotated during that time.

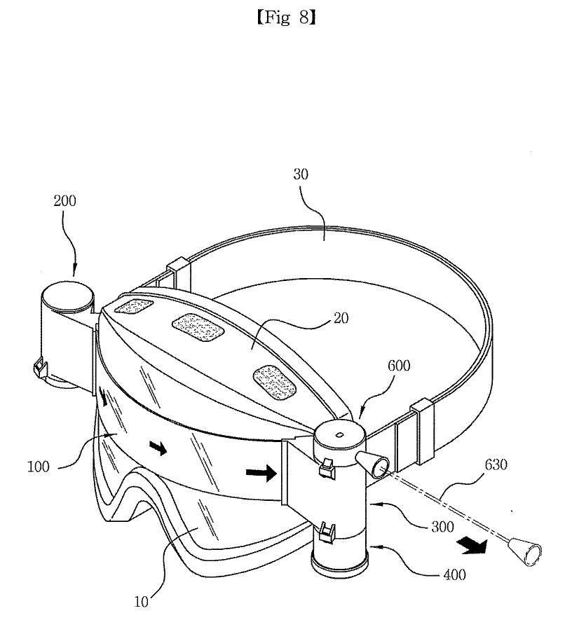

Since the drive part 400 and the manipulating part 500 are connected electrically to each other, malfunctions may occur thereon, and also, they may be not operated by the low charge of their batteries. To prevent such problems, as shown in FIG. 8, the goggles according to the present invention further includes an auxiliary part 600 adapted to be coupled to the film return part 300 so as to roll the film 100 in a manually operated manner.

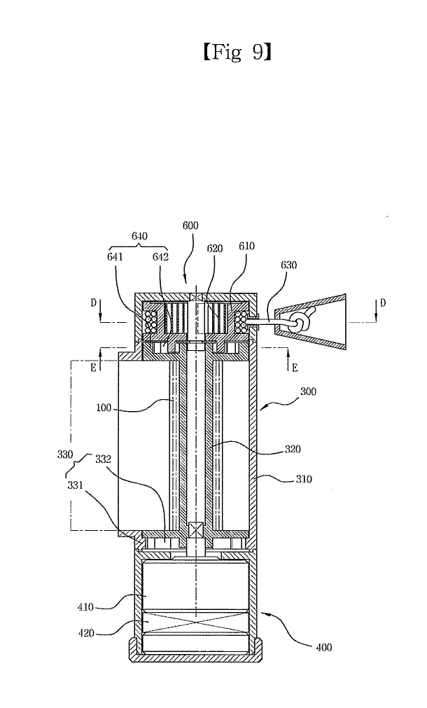

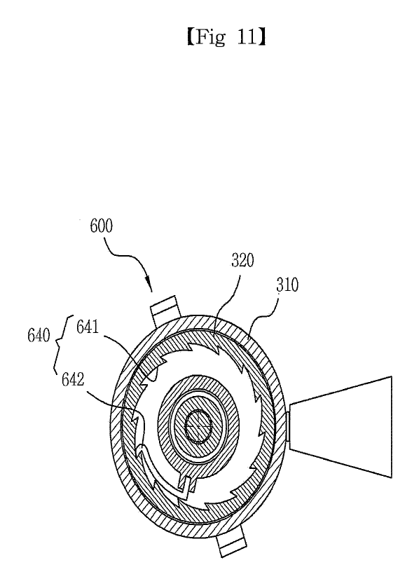

As shown in FIGS. 9 and 11, the auxiliary part 600 includes a reel 610, a spring 620, a pulling string 630, and a one-way clutch 640.

The reel 610 is shaft-coupled coaxially to the winding roll 320 of the film return part 300. That is, the reel 610 is shaft-coupled at the inside of the case 310 so as to be rotated separately from the winding roll 320.

The spring 620 is disposed inside the reel 610 and serves to rotate the reel 610 forwardly and reversely by means of its own elastic force. That is, the spring 620 is wound to increase the elastic force while the reel 610 is rotated forwardly to roll the film 100, and contrarily, after the film 100 is rolled, the reel 610 is rotated reversely by means of the elastic force of the spring 620 such that the spring 620 is returned to its original shape.

The pulling string 630 is adapted to be wound along the outer periphery of the reel 610 and has a hand grip disposed at the end thereof. That is, if the pulling string 630 is forcedly pulled in the state of being wound on the reel 610, the reel 610 is rotated forwardly to compress the spring 620, and contrarily, if pulling of the pulling string 630 is released, the spring 620 is returned and wound on the reel 610.

The one-way clutch 640 is disposed between the reel 610 and the winding roll 320 of the film return part 300 and serves to rotate the winding roll 320 in one direction.

The one-way clutch 640 includes a latch gear 641 disposed along the inner surface of the upper periphery of the winding roll 320 and an elastic latch 642 adapted to be coupled to the reel 610 in such a manner as to be locked to the latch gear 641 when the reel 610 is rotated forwardly and contrarily to be unlocked from the latch gear 641 when the reel 610 is rotated reversely.

As set forth in the foregoing, there are provided the goggles with the lens protection film wherein while the film covering the front surfaces of the lenses is rolled to one side of the goggles, the foreign materials sticking to the surfaces of the lenses are removed to allow the wearer's view to be clearly ensured.

According to the present invention, further, the film is automatically wound by means of the drive part remotely controlled by the manipulating part, thereby allowing the foreign materials on the lenses to be removed safely and simply.

Additionally, the auxiliary part is provided to wind the film in a manually operated manner, such that when the drive part does not work, it is rapidly replaced with the auxiliary part.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. Goggles having lenses protecting a wearer's eyes, a frame coupled to the lenses in such a manner as to be brought into close contact with the wearer's face, and a band coupled to both sides of the frame in such a manner as to be worn around the wearer's head, the goggles comprising:
film made of a transparent material so as to provide a clear view in a state of covering the front surfaces of the lenses and having an elongated belt-like shape so as to be rolled;
a film supply part adapted to be coupled to one side of the goggles so as to unroll the film toward the front of the lenses in a state of winding the film thereinto;
a film return part adapted to be coupled to the other side of the goggles so as to roll the film unrolled to the front of the lenses thereinto;
a drive part adapted to be coupled to the film return part so as to generate power to automatically roll the film; and
a manipulating part adapted to be connected to the drive part with wire or without wire so as to remotely control an operation of the drive part.

2. The goggles according to claim 1, wherein each of the film supply part and the film return part comprises:
a case adapted to be coupled to one side of the goggles and having entrance and exit formed in an opposite direction thereto;
a winding roll disposed inside the case and adapted to be rotated forwardly to roll or unroll the film thereonto or therefrom; and
a one-way clutch adapted to prevent the winding roll from being rotated reversely.

3. The goggles according to claim 2, wherein the one-way clutch comprises a latch gear disposed at the inside of the case and an elastic latch adapted to be coupled to the winding roll in such a manner as to be locked to the latch gear when the winding roll is rotated forwardly and unlocked from the latch gear when the winding roll is rotated reversely.

4. The goggles according to claim 1, wherein the drive part has a motor adapted to be coupled to the winding roll and a receiver module adapted to receive a wireless signal and to operate the motor, and the manipulating part has a switch adapted to control the activation of the drive part and a transmitter module adapted to transmit the wireless signal in accordance with the manipulation of the switch.

5. The goggles according to claim 1, further an auxiliary part adapted to be coupled to the film return part so as to roll the film in a manually operated manner.

6. The goggles according to claim 5, wherein the auxiliary part comprises:
a reel shaft-coupled coaxially to the winding roll of the film return part;
a spring disposed inside the reel and adapted to rotate the reel forwardly and reversely by means of the elastic force thereof;
a pulling string adapted to be wound along the outer periphery of the reel and having a hand grip disposed at the end thereof; and
a one-way clutch disposed between the reel and the winding roll of the film return part and adapted to rotate the winding roll in one direction.

7. The goggles according to claim 6, wherein the one-way clutch comprises:
a latch gear disposed along the inner surface of the upper periphery of the winding roll; and
an elastic latch adapted to be coupled to the reel in such a manner as to be locked to the latch gear when the reel is rotated forwardly and unlocked from the latch gear when the reel is rotated reversely.
